SEQUENCE LISTING (1 sequence)

SEQ ID NO: 1 - protein, 127 aa, Homo sapiens
MSFSGKYQLQSQENFEAFMKAIGLPEELIQKGKDIKGVSEIVQNGKHFKFTITAGSKVIQNEFTVGEECELETMTGEKVKTVVQLEGDNKLVTTFKNIKSVTELNGDIITNTMTLGDIVFKRISKRI

(19) United States
(12) Patent Application Publication
(10) Pub. No.: US 2022/0034909 A1
(43) Pub. Date: Feb. 3, 2022

(54) METHOD AND KIT FOR QUANTIFYING LIVER-TYPE FATTY ACID BINDING PROTEIN, METHOD AND KIT FOR TESTING FOR KIDNEY DISEASES, AND COMPANION DIAGNOSTIC DRUG

(71) Applicant: CMIC HOLDINGS CO., LTD., Tokyo (JP)

(72) Inventors: Keiichi OHATA, Tokyo (JP); Takeshi SUGAYA, Tokyo (JP); Tsuyoshi OIKAWA, Tokyo (JP)

(73) Assignee: CMIC HOLDINGS CO., LTD., Tokyo (JP)

(21) Appl. No.: 17/280,602

(22) PCT Filed: Sep. 27, 2019
(86) PCT No.: PCT/JP2019/038257

§ 371 Date: Mar. 26, 2021

(30) Foreign Application Priority Data
Sep. 28, 2018 (JP) 2018-185920

(51) Int. Cl. G01N 33/68

(52) U.S. Cl. CPC G01N 33/6893; G01N 2800/34

(57) ABSTRACT

Provided are a method and a kit for quantifying L-FABP or oxidized L-FABP in any sample, a method and a kit for testing for kidney diseases on the basis of the quantifying result of L-FABP or oxidized L-FABP in urine of a subject, and a companion diagnostic drug. This method for quantifying liver type fatty acid binding protein includes a step for promoting an antigen-antibody reaction, and quantifying the liver type fatty acid binding protein under a condition in which the measurement sensitivity of oxidized liver type fatty acid binding protein is higher than that of unoxidized liver type fatty acid binding protein.

### TECHNICAL FIELD

The present invention relates to a method for quantifying L-type fatty acid binding protein in a sample, a quantification kit for the same, a method for testing kidney diseases, a test kit for the same, and a companion diagnostic agent.

### BACKGROUND ART

L-type fatty acid binding protein (hereinafter, simply referred to as “L-FABP”) exists in cytoplasm in e.g. liver and proximal convoluted tubule cells in kidney. The amount thereof excreted into urine increases in response to ischemia due to renal tubular disorders or oxidative stress in kidney (e.g. Non-Patent Document 1). Therefore, kidney diseases can be tested based on the detection of the total amount of L-FABP protein derived from kidney tissue in urine (e.g. Patent Document 1). It is known that L-FABP is stabilized in a form in which a β-barrel structure having two antiparallel β-sheets running straight has a lid formed by two α-helices, and L-FABP protein binds to two molecules of a free fatty acid (e.g. Non-Patent Document 2).

The structure of L-FABP is changed due to the modifications of methionine oxidation, and the inner region of L-FABP molecules is exposed (e.g. Non-Patent Document 3). Accordingly, it is known that in the measurement using an antigen-antibody reaction such as ELISA, the antibody-binding capacity is changed by using an antibody which binds to the inner region of L-FABP molecules, and measured values are largely changed. In addition, it has been reported that the modifications of methionine oxidation in L-FABP occur due to e.g. a treatment with 2,2′-azobis2-amidinopropane (hereinafter, abbreviated to “AAPH”) and air oxidation (Patent Documents 2 to 4).

Patent Document 5 discloses a method for improving the sensitivity of immunoassay, i.e. the measurement sensitivity of proteins in urine, a subject to be measured, by adding one or two of compounds consisting of reducing agents (such as glutathione, cysteine and penicillamine), chaotropic reagents (such as urea and guanidine) and surfactants (such as sodium n-dodecylbenzene sulfonate) as a denaturant to a urine specimen, and pretreating the urine specimen using these compounds. In Patent Document 5, L-FABP is provided as an example of the proteins in urine; however, the detection of L-WASP is not specifically described. In addition, Patent Document 6 discloses a method for promoting agglutination based on specific reactions without causing spontaneous agglutination of carrier particles by using an organic amine compound. Patent Document 7 discloses a method for improving measurement sensitivity by bringing a compound having a partial structure, NH₂—C═N—, and a cyclic structure in a molecule thereof such as a benzamidine derivative into contact with L-FABP in a specimen. However, the methods are not described as a method for evaluating the oxidation state of L-FABP using an antibody which binds to the inner region of L-FABP molecules.

Patent Document 1: Japanese Unexamined Patent Application, Publication No. H11-242026

### Patent Document 2: Japanese Patent No. 6174778

### Patent Document 3: Japanese Patent No. 6218983

### Patent Document 4: Japanese Patent No. 6059388

### Patent Document 5: Japanese Unexamined Patent Application, Publication No. 2014-85208

Patent Document 6: PCT International Publication No. WO2007/074860
Patent Document 7: PCT International Publication No. WO2016/136863

### Non-Patent Document 1: Kamijo, A. et al.: J Lab Clin Med, 143: 23-30, 2004

### Non-Patent Document 2: Cai, J., et al.: Biophys J, 102: 2585-2594, 2012. Non-Patent Document 3: Yan, J., et al.: J Lipid Res, 50: 2445-2454, 2009.

### DISCLOSURE OF THE INVENTION

### Problems to be Solved by the Invention

The present invention was made in view of such actual circumstances of prior art, and an object thereof is to provide a method for quantifying L-FABP or oxidized L-FABP in any sample, a quantification kit for the same, a method for testing kidney diseases based on the results of quantifying L-FABP or oxidized L-FABP in urine of a subject, a test kit for the same, and a companion diagnostic agent.

### Means for Solving the Problems

As a result of repeated diligent researches to solve the above problems, the present inventors found that a condition that the measurement sensitivity of oxidized L-FABP be relatively higher than that of unoxidized L-FABP and the measurement sensitivity of oxidized L-FABP be also absolutely high could be realized by adequately promoting an antigen-antibody reaction. The present inventors also found that the oxidation rate of L-FABP in urine is different between patients with chronic kidney disease (CKD) and patients with acute kidney injury (AKI). The present invention was completed based on the above knowledge. That is, the present invention is as follows.

<1> A method for quantifying L-type fatty acid binding protein, including a step of promoting an antigen-antibody reaction, and quantifying L-type fatty acid binding protein under a condition that the measurement sensitivity of oxidized L-type fatty acid binding protein is higher than the measurement sensitivity of unoxidized L-type fatty acid binding protein.
<2> The method according to <1>, in which the condition is a condition formed by a treatment with a chaotropic reagent or an organic amine compound.
<3> The method according to <1> or <2>, further including a step of quantifying L-type fatty acid binding protein under a condition that a difference in measurement sensitivity between oxidized L-type fatty acid binding protein and unoxidized L-type fatty acid binding protein is smaller than the difference in measurement sensitivity under the condition that the measurement sensitivity of oxidized L-type fatty acid binding protein is higher than the measurement sensitivity of unoxidized L-type fatty acid binding protein.
<4> The method according to <3>, in which the condition that a difference in measurement sensitivity is smaller is a condition formed by a denaturing treatment of the L-type fatty acid binding protein in a sample by a surfactant.
<5> The method according to <3> or <4>, further including a step of calculating an oxidation rate, which substantially corresponds to a rate of the oxidized L-type fatty acid binding protein to the L-type fatty acid binding protein in a sample, based on a measured value of the L-type fatty acid binding protein under the condition that the difference in measurement sensitivity is small and a measured value under the condition that the measurement sensitivity of the oxidized L-type fatty acid binding protein is high.
<6> A quantification kit, used for the method according to any one of <1> to <5>, the kit including a substance which can quantify L-type fatty acid binding protein.
<7> A method for testing a kidney disease, including a step of promoting an antigen-antibody reaction, and quantifying L-type fatty acid binding protein in urine of a subject under a condition that measurement sensitivity of oxidized L-type fatty acid binding protein is higher than measurement sensitivity of unoxidized L-type fatty acid binding protein.
<8> A method for testing a kidney disease, including a step of quantifying an amount of oxidized L-type fatty acid binding protein in urine of a subject or a parameter value which correlates therewith after promoting an antigen-antibody reaction.
<9> The method according to <8>, in which the quantification is quantification under a condition that measurement sensitivity of oxidized L-type fatty acid binding protein is higher than measurement sensitivity of unoxidized L-type fatty acid binding protein.
<10> The testing method according to <7> or <9>, in which the condition is a condition formed by a treatment with a chaotropic reagent or an organic amine compound.
<11> The method according to <7>, <9> or <10>, further including a step of quantifying the L-type fatty acid binding protein under a condition that a difference in measurement sensitivity between the oxidized L-type fatty acid binding protein and the unoxidized L-type fatty acid binding protein is smaller than the difference in measurement sensitivity under the condition that the measurement sensitivity of the oxidized L-type fatty acid binding protein is higher than the measurement sensitivity of the unoxidized L-type fatty acid binding protein.
<12> The method according to <11>, in which the condition that a difference in measurement sensitivity is smaller is a condition formed by a denaturing treatment of the L-type fatty acid binding protein in urine by a surfactant.
<13> The method according to <11> or <12>, further including a step of calculating an oxidation rate, which substantially corresponds to a rate of the oxidized L-type fatty acid binding protein to the L-type fatty acid binding protein in urine, based on a measured value of the L-type fatty acid binding protein under the condition that a difference in measurement sensitivity is small and a measured value under the condition that the measurement sensitivity of the oxidized L-type fatty acid binding protein is high.
<14> A method for testing a kidney disease based on an amount of oxidized L-type fatty acid binding protein in a subject or a parameter value which correlates therewith, the method including a step of:
comparing
a known normal range of an amount of the oxidized L-type fatty acid binding protein or a parameter value which correlates therewith, or a known range of an amount of the oxidized L-type fatty acid binding protein in a kidney disease or a parameter value which correlates therewith, and
an amount of the oxidized L-type fatty acid binding protein in urine of a subject or a parameter value which correlates therewith; and
determining in which range the amount in the subject or the parameter value which correlates therewith is included.
<15> A test kit, used for the method according to any one of <7> to <14>, the kit including a substance which can quantify the L-type fatty acid binding protein or the oxidized L-type fatty acid binding protein.
<16> A companion diagnostic agent, used for the method according to any one of <7> to <14>, the agent including a substance which can quantify the L-type fatty acid binding protein or the oxidized L-type fatty acid binding protein.
<17> A kidney disease marker, used as a target to be quantified in the method according to any one of <7> to <14>, the marker including L-type fatty acid binding protein or oxidized L-type fatty acid binding protein.
<18> The method according to any one of <1> to <5>, including a step of collecting a sample from a subject and a step of detecting the L-type fatty acid binding protein in the sample.
<19> The method according to any one of <1> to <5> and <7> to
<14>, including a step of collecting urine from a subject and a step of detecting the L-type fatty acid binding protein in the urine, and also including at least one step selected from the group consisting of the following (A) and (B1) to (B4):
(A) a step of comparing a known normal range of an amount of the oxidized L-type fatty acid binding protein or a parameter value which correlates therewith, or a known range of an amount of the oxidized L-type fatty acid binding protein in a kidney disease or a parameter value which correlates therewith, and an amount of the oxidized L-type fatty acid binding protein in urine of a subject or a parameter value which correlates therewith, and determining in which range the amount in the subject or the parameter value which correlates therewith is included,
(B1) a step of comparing an amount of oxidized L-type fatty acid binding protein in a healthy subject or a parameter value which correlates therewith, and an amount of oxidized L-type fatty acid binding protein in a subject or a parameter value which correlates therewith, and determining that the subject is contracted with a chronic kidney disease when the above value of the latter is detected to be significantly higher than the above value of the former, in which the above amount of oxidized L-type fatty acid binding protein in the healthy subject or the value may be an amount or a value when the subject was previously healthy,
(B2) a step of comparing an amount of oxidized L-type fatty acid binding protein in a healthy subject or a parameter value which correlates therewith, and an amount of oxidized L-type fatty acid binding protein in a subject or a parameter value which correlates therewith, and determining that the subject is contracted with an acute kidney disease when the above value of the latter is detected to be significantly lower than the above value of the former, in which the above amount of oxidized L-type fatty acid binding protein in the healthy subject or the value may be an amount or a value when the above subject was previously healthy,
(B3) a step of comparing an amount of oxidized L-type fatty acid binding protein in a patient with an acute kidney disease or a parameter value which correlates therewith, and an amount of L-type fatty acid binding protein in a subject, and determining that the subject is contracted with a chronic kidney disease, when the above value of the latter is detected to be significantly higher than the above value of the former, in which the above amount of oxidized L-type fatty acid binding protein in a patient with an acute kidney disease or the value may be an amount or a value when the above subject was previously contracted with an acute kidney disease, and
(B4) a step of comparing an amount of oxidized L-type fatty acid binding protein in a patient with a chronic kidney disease or a parameter value which correlates therewith, and an amount of L-type fatty acid binding protein in a subject, and determining that the subject is contracted with an acute kidney disease, when the above value of the latter is detected to be significantly lower than the above value of the former, in which the above amount of oxidized L-type fatty acid binding protein in the patient with a chronic kidney disease or the value may be an amount or a value when the above subject was previously contracted with a chronic kidney disease.
<20> The method according to any one of <7> to <14>, including a method for diagnosing kidney diseases.
<21> A method for treating or preventing a kidney disease, including the method according to any one of <7> to <14> above, and a step of administering a therapeutic agent or preventive medicine for a kidney disease determined by the method to a subject.
<22> The method according to <21>, in which the above therapeutic agent or preventive medicine for a kidney disease includes at least one drug selected from the group consisting of therapeutic agents or preventive medicines for chronic kidney disease and therapeutic agents or preventive medicines for acute kidney disease.
<23> The method according to any one of <1> to <5> and <7> to <14>, in which under the above condition that the measurement sensitivity of oxidized L-type fatty acid binding protein is higher than the measurement sensitivity of unoxidized L-type fatty acid binding protein, the above oxidized L-type fatty acid binding protein is L-type fatty acid binding protein oxidized by 2,2′-azobis 2-amidinopropane and the above unoxidized L-type fatty acid binding protein is L-type fatty acid binding protein, not being oxidized by 2,2′-azobis 2-amidinopropane;
the above oxidized L-type fatty acid binding protein is L-type fatty acid binding protein oxidized by an arbitrary oxidizing agent or air and the above unoxidized L-type fatty acid binding protein is L-type fatty acid binding protein, being oxidized by neither the arbitrary oxidizing agent nor air; or the above oxidized L-type fatty acid binding protein is oxidized L-type fatty acid binding protein in an arbitrary manner, and the above unoxidized L-type fatty acid binding protein is L-type fatty acid binding protein, not being oxidized in the arbitrary manner.

### Effects of the Invention

According to the present invention, it is possible to provide a method for quantifying L-FABP or oxidized L-FABP in any sample and a quantification kit for the same. According to the present invention, it is also possible to provide a method for being able to test kidney diseases such as chronic kidney disease and acute kidney injury based on the results of quantifying L-FABP or oxidized L-FABP in urine of a subject, a test kit for the same, and a companion diagnostic agent.

### BRIEF DESCRIPTION OF THE DRAWINGS

FIG. 1 are graphs showing the results of Reference Example 1; and

FIG. 2 are graphs showing the results of Example 1.

### PREFERRED MODE FOR CARRYING OUT THE INVENTION

Embodiments of the present invention will now be described in detail. It should be noted, however, that the present invention is not limited to the embodiments described below, and can be performed with appropriate modifications within the objects of the present invention.

### (L-FABP)

The amino acid sequence and gene sequence of L-FABP have been already reported (Veerkamp and Maatman, Prog. Lipid Res., 34:17-52, 1995). SEQ ID NO:1 shows the amino acid sequence of wild-type human L-FABP. Even mutant proteins having substitutions, insertions, deletions and the like on the amino acid sequence of wild-type human L-type fatty acid binding protein shown in SEQ ID NO:1 of the sequence listing all can fall within L-type fatty acid binding protein if the mutation demonstrates high conservation in the 3-dimensional structure of wild-type human L-type fatty acid binding protein. The side chains of amino acids, which are constituents of the proteins, vary in hydrophobicity, charge, size and the like. Several relationships having high conservation in a sense that there is not a substantial effect on the three-dimensional structure (also referred to as conformation) of the whole protein are known experientially or by physicochemical actual measurement. Examples of substitutions of amino acid residues include glycine (Gly) and proline (Pro), Gly and alanine (Ala) or valine (Val), leucine (Leu) and isoleucine (Ile), glutamic acid (Glu) and glutamine (Gin), aspartic acid (Asp) and asparagine (Asn), cysteine (Cys) and threonine (Thr), Thr and serine (Ser) or Ala, lysine (Lys) and arginine (Arg) and the like.

The method for obtaining the above L-FABP is not particularly restricted, and the L-FABP may be a protein synthesized by chemical synthesis or a recombinant protein produced by a genetic engineering technique.

### <<Method for Quantifying L-FABP>>

The first aspect of the present invention is a method for quantifying L-FABP, including a step of promoting an antigen-antibody reaction, and quantifying L-FABP under a condition that the measurement sensitivity of oxidized L-type fatty acid binding protein (hereinafter, simply referred to as “oxidized L-FABP”) be higher than the measurement sensitivity of unoxidized L-type fatty acid binding protein (hereinafter, simply referred to as “unoxidized L-FABP”). The method for quantifying L-FABP according to the first aspect may or may not include a step of collecting a sample from a subject, and may or may not include a step of detecting L-type fatty acid binding protein in the sample. The sample containing L-FABP may be any sample, and examples thereof include urine, blood, sweat and the like. The sample is preferably urine.

In the method for quantifying L-FABP according to the first aspect, the sample may or may not include unoxidized L-FABP, and may include a mixture of oxidized L-FABP and unoxidized L-FABP. The sample preferably include a mixture of oxidized L-FABP and unoxidized L-FABP or oxidized L-FABP.

In L-FABP, methionine at residues 19, 74 and 113 in SEQ ID NO:1 can be oxidized, and it can be said that the above oxidized L-FABP is L-FABP in which at least any one of methionine at residues 19, 74 and 113 is oxidized. In particular because it is thought that changes in measured values using an anti-L-FABP antibody are dominated by oxidation of methionine at residues 19 and 113, L-FABP in which at least either of methionine at residues 19 and 113 is oxidized is preferred. Examples of methods for measuring e.g. detecting or quantifying L-FABP include assays using e.g. enzyme immunoassay (EIA, ELISA), fluorescence enzyme immunoassay (FLEIA), chemiluminescent enzyme immunoassay (CLEIA), chemiluminescent immunoassay (CLIA), electrochemiluminescence immunoassay (ECLIA), fluorescent antibody method (FA), radioimmunoassay (RIA), western blotting (WB), or immunoblotting. The method for measuring e.g. detecting or quantifying L-FABP is preferably the measurement using an anti-L-FABP antibody.

The anti-L-FABP antibody used is not particularly restricted as long as it can recognize L-FABP, and it may be a known antibody or an antibody which will be developed in the future. In the case of the measurement using an anti-L-FABP antibody, an antibody which recognizes a site exposed to the outside by the above methionine oxidation is further preferred. In addition, an anti-oxidized L-FABP antibody which does not recognize unoxidized L-FABP but can specifically recognize oxidized L-FABP can be also used; however, the above condition in the method for quantifying L-FABP according to the first aspect does not include such antibody-dependent condition.

The above “condition that the measurement sensitivity of oxidized L-FABP be higher than the measurement sensitivity of unoxidized L-FABP” may be satisfied with any one or at least one selected from the group consisting of “the above oxidized L-FABP is L-FABP oxidized by AAPH and the above unoxidized L-FABP is L-FABP, not being oxidized by AAPH”, “the above oxidized L-FABP is L-FABP oxidized by an arbitrary oxidizing agent or air and the above unoxidized L-FABP is L-FABP, being oxidized by neither the arbitrary oxidizing agent nor air” and “the above oxidized L-FABP is oxidized L-FABP in an arbitrary manner, and the above unoxidized L-FABP is L-FABP, not being oxidized in the arbitrary manner”, or may be satisfied with other conditions. Specifically, the quantification under the above condition is more preferably quantification under a condition that, for example, when oxidized recombinant L-FABP treated with 50 mM AAPH at 37° C. for 60 minutes, and unoxidized recombinant L-FABP, which is not treated, are subjected to ELISA using the antibody from “RENISCHEM L-FABP ELISA High Sensitivity Kit” (manufactured by CMIC HOLIDNGS CO., LTD.) and the color intensity (OD 450 nm) of a labeled antibody is measured, the measurement sensitivity of oxidized L-FABP be 1.4 times or more (preferably 1.5 times or more, more preferably 1.8 times or more, and further preferably 2.0 times or more) higher than that of unoxidized L-FABP at a concentration of 25 ng/ml. The upper limit of the rate of measurement sensitivity is not particularly restricted, and examples thereof include 6 times or less or 4 times or less. As used herein, the “unoxidized recombinant L-FABP, which is not treated” means, when after a treatment with at least one of 1000 mM benzamidine hydrochloride or 1500 mM guanidinium chloride at 25° C. for 10 minutes, ELISA is performed using the antibody from “RENISCHEM L-FABP ELISA High Sensitivity Kit” and the color intensity (OD 450 nm) of a labeled antibody is measured, L-FABP having a color intensity of 0.7 times or less that of oxidized L-FABP treated with 50 mM AAPH at 37° C. for 60 minutes at a concentration of 25 ng/ml.

More particularly, the above measurement method is preferably sandwich ELISA using two antibodies combined, which have different recognition sites to the antigen (L-FABP). It is preferred that as the two antibodies having different recognition sites, one be used as a solid-phase antibody, which is bound to the surface of microplate wells, and the other be used as a labeled antibody for detection or quantification. The label in the above labeled antibody is not particularly restricted, and examples thereof include enzyme labels such as peroxidase label, fluorescent labels, UV labels, radiation labels and the like.

Examples of the antibodies having different recognition sites to the antigen (L-FABP) include antibodies including an antibody selected from the group consisting of anti-L-FABP antibodies clone 1, clone 2, clone L and clone F (e.g. Patent Documents 2 to 4), and the antibodies are preferably a combination including an anti-L-FABP antibody clone L, or a combination including an anti-L-FABP antibody clone 2, more preferably a combination including an anti-L-FABP antibody clone L, further preferably a combination in which an anti-L-FABP antibody clone L is used as a solid-phase antibody and any anti-L-FABP antibody is used as a labeled antibody, and particularly preferably a combination in which an anti-L-FABP antibody clone L is used as a solid-phase antibody and an anti-L-FABP antibody clone 2 is used as a labeled antibody. Examples of commercial products of kits for quantifying L-FABP using sandwich ELISA include “RENISCHEM L-FABP ELISA TMB Kit” (manufactured by CMIC HOLIDNGS CO., LTD.), “RENISCHEM L-FABP ELISA High Sensitivity Kit” (manufactured by CMIC HOLIDNGS CO., LTD.) and the like.

In the case of e.g. quantification using an anti-L-FABP antibody, for example, an antigen-antibody reaction is promoted, and also the physicochemical characteristics of L-FABP are mildly changed under the above condition that the measurement sensitivity of oxidized L-FABP be higher to promote the reaction of L-FABP and the antibody, and denaturation does not proceed to an extent that the conformation of L-FABP is lost. Because of this, absolute measurement sensitivity can be increased while maintaining or enhancing characteristics in that the measurement sensitivity of oxidized L-FABP is higher than the measurement sensitivity of unoxidized L-FABP. Such condition can be formed by using various protein denaturants in combination with adequate use conditions, and a substance with a mild protein denaturing action is preferably used because the degree of freedom of use conditions increases. However, when using a substance with a strong protein denaturing action (e.g. sodium dodecyl sulfate (SDS)), the degree of freedom of use conditions is correspondingly reduced (restrictions such as a low concentration, a low temperature and a short period of time are placed), but the above condition can be formed. From this viewpoint, the so-called immunoagglutination promoter is preferred, and specifically a chaotropic reagent or an organic amine compound is more preferred. As described in Reference Example 1 below, the measurement sensitivity of oxidized L-FABP after a treatment with an immunoagglutination promoter under an adequate condition absolutely significantly increases and is also relatively higher than that of unoxidized L-FABP. Therefore, oxidized L-FABP in a sample can be quantified by comparing a measured value obtained by using an anti-L-FABP antibody after a treatment with an immunoagglutination promoter, and a measured value obtained by using an anti-L-FABP antibody without the above treatment (preferably, a measured value under a condition that a difference in measurement sensitivity between oxidized L-FABP and unoxidized L-FABP be small described below).

Examples of the immunoagglutination promoter include chaotropic reagents, organic amine compounds, reducing agents (such as glutathione, cysteine and penicillamine), surfactants (such as sodium n-dodecylbenzene sulfonate), or substances having the same effect, and the like, and a chaotropic reagent or an organic amine compound is preferred. In the method for quantifying oxidized L-FABP according to the first aspect, the above quantification is more preferably quantification of L-FABP after a treatment with a chaotropic reagent or an organic amine compound. The anti-L-FABP antibody used for measurement is the same as above.

As specific examples of the above chaotropic reagent or organic amine compound, at least one selected from urea, 2-amino-2-thiazoline hydrochloride, benzamidine hydrochloride, benzylamine hydrochloride, guanidine hydrochloride, aminopyrine, antipyrine, 4-aminoantipyrine, o-phenylenediamine dihydrochloride, p-anisidine hydrochloride, diphenhydramine hydrochloride, 2,4-diaminoanisole dihydrochloride, pyridine hydrochloride, p-phenylenediamine hydrochloride, aminoguanidine hydrochloride and betaine hydrochloride is preferably used. Among these, benzamidine hydrochloride, benzylamine hydrochloride, and 2-amino-2-thiazoline hydrochloride are further preferred. A compound represented by the following formula (A) or a salt or an ester thereof, and a compound represented by the following formula (B) or a salt thereof can be also preferably used.

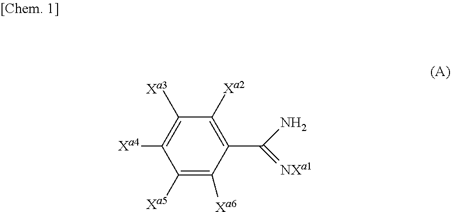

(In the formula (A), X^{a1} is a hydrogen atom, a hydroxyl group or an alkyl group, and X^{a2} to X^{a6} are each independently a hydrogen atom, a halogen atom, an alkyl group, a hydroxyl group, a carboxy group, an amino group or —SX^{a7} (X^{a7} is a hydrogen atom, a hydroxyl group or an alkyl group. When a plurality of X^{a7} exist, the groups may be the same or different).) Examples of the above alkyl group include linear or branched alkyl groups, and a C1-3 alkyl group is preferred.

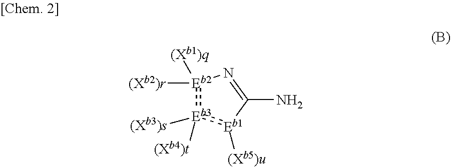

(In the formula (B), X^{b1} to X^{b4} are each independently a hydrogen atom, a halogen atom, an alkyl group, an amino group, a phenyl group which may be substituted with a halogen atom, or —SX^{b6} (X^{b6} is a hydrogen atom, a hydroxyl group or an alkyl group. When a plurality of X^{b6} exist, the groups may be the same or different.) Here, when both X^{b1} and X^{b2} exist, they may be bound to each other to form a carbonyl group, and when both X^{b3} and X^{b4} exist, they may be bound to each other to form a carbonyl group. X^{b5} is a hydrogen atom, a halogen atom or an alkyl group,
E^{b1} is a nitrogen atom or a sulfur atom,
E^{b2} and E^{b3} are each independently a carbon atom or a nitrogen atom,
q, r, s, t and u are each independently 0 or 1,
the double dashed line between E^{b1} and E^{b3} and the double dashed line between E^{b2} and E^{b3} are each independently a single bond or a double bond, and the values of the above q, r, s, t and u, and the bonds of the double dashed line between E^{b1} and E^{b3} and the double dashed line between E^{b2} and E^{b3} are values and bonds appropriately determined depending on the valences of E^{b1} to E^{b3})
Examples of the above alkyl group include linear or branched alkyl groups, and a C1-3 alkyl group is preferred.

It should be noted that the salts of organic amine compounds are not particularly restricted, and include hydrosulfates, nitrates, hydrobromides, hydrofluorides, hydrofluoroborides, oxalates, lactates, adipates, tartrates, hydroiodides, toluenesulfonates, malonates, bicarbonates and the like, and the salt can be appropriately selected in view of e.g. handleability and ease of access as a reagent in addition to the effects of the present invention.

Examples of the treatment with an immunoagglutination promoter such as the above chaotropic reagent or organic amine compound include a method in which the treatment is carried out by an immunoagglutination promoter in an adequate concentration (e.g. 10 mM to 3000 mM) at room temperature (e.g. 25° C.) or under a heating condition (e.g. 35° C. or lower) for an adequate time (e.g. 5 to 60 minutes). From the viewpoint of achieving a condition that the measurement sensitivity of oxidized L-FABP be higher than that of unoxidized L-FABP and from the viewpoint that the a difference in measurement sensitivity is small at a range of 35° C. or lower, preferred is a method in which the treatment is carried out by an immunoagglutination promoter in any concentration at room temperature or under a heating condition of 35° C. or lower, more preferred is a method in which the treatment is carried out by an immunoagglutination promoter in any concentration at room temperature or under a heating condition of 33° C. or lower, further preferred is a method in which the treatment is carried out by an immunoagglutination promoter in any concentration at room temperature or under a heating condition of 30° C. or lower, particularly preferred is a method in which the treatment is carried out by an immunoagglutination promoter in any concentration at room temperature or under a heating condition of 28° C. or lower, and most preferred is a method in which the treatment is carried out by an immunoagglutination promoter in any concentration at room temperature (e.g. 25° C.). Typically, the treatment is carried out by 1000 mM benzamidine hydrochloride or 1500 mM guanidinium chloride at 25° C. for 10 minutes. The immunoagglutination promoters such as the above chaotropic reagent and organic amine compound may be used individually or two or more immunoagglutination promoters may be used in combination.

Examples of the treatment with a surfactant such as SDS include a method in which the treatment is carried out by a surfactant in an adequate low concentration (e.g. 0.12% weight/volume or less) at a low temperature (e.g. 25° C. or lower) for an adequate short time (e.g. less than 4 minutes).

The method for quantifying L-FABP according to the first aspect preferably further includes a step of quantifying the above L-FABP under a condition that a difference in measurement sensitivity between oxidized L-FABP and unoxidized L-FABP is smaller than the difference in measurement sensitivity under the condition that the measurement sensitivity of the oxidized L-FABP is higher than the measurement sensitivity of the unoxidized L-FABP. Examples of the condition that a difference in measurement sensitivity between oxidized L-FABP and unoxidized L-FABP is small include a condition that when oxidized recombinant L-FABP treated with 50 mM AAPH at 37° C. for 60 minutes, and unoxidized recombinant L-FABP, which is not treated, are subjected to ELISA using the antibody from “RENISCHEM L-FABP ELISA High Sensitivity Kit” (manufactured by CMIC HOLIDNGS CO., LTD.) and the color intensity (OD 450 nm) of a labeled antibody is measured, the measurement sensitivity of oxidized L-FABP is 0.8 times or more and less than 1.4 times (preferably 0.9 times or more and 1.25 times or less) that of unoxidized L-FABP at a concentration of 25 ng/ml.

Under the above condition that a difference in measurement sensitivity is small, the conformation is modified by cleaving e.g. a hydrogen bond and a disulfide bond with the primary structure of L-FABP maintained. Because of this, L-FABP can be detected or quantified at a high sensitivity and specifically without influence by the oxidation state of L-FABP even when an antibody is bound to the inner region of L-FABP molecules. Such condition can be formed by using various protein denaturants in combination with adequate use conditions, and a substance with a strong protein denaturing action is preferably used because the degree of freedom of use conditions increases. However, when using a substance with a mild protein denaturing action (e.g. the above immunoagglutination promoter), the degree of freedom of use conditions is correspondingly reduced (restrictions such as a high concentration, a high temperature and a long period of time are placed), but the above condition can be formed. From this viewpoint, a surfactant is preferred, and specifically sodium dodecyl sulfate (SDS) is preferred. As used herein “unoxidized recombinant L-FABP, which is not treated” is as described above. Examples of the above denaturing treatment include a method in which the treatment is carried out by a surfactant in an adequate concentration (may be e.g. 0.2% weight/volume (w/v %) to 10% weight/volume, preferably 0.4% weight/volume (w/v %) or more, 0.5% weight/volume (w/v %) or more, or 0.7% weight/volume (w/v %) or more) for an adequate time (e.g. 5 to 60 minutes) at room temperature (e.g. 25° C.) or under a heating condition (e.g. 37° C.). Typically, the denaturing treatment is carried out by 1 w/v % SDS at 25° C. for 10 minutes.

Examples of the treatment with an immunoagglutination promoter include a method in which the treatment is carried out by an immunoagglutination promoter in an adequate high concentration (e.g. 3500 mM) for an adequate long time (e.g. 80 minutes) under a heating condition (e.g. 37° C. or higher).

In the description and claims, the “oxidation rate of L-FABP” can be defined as the rate of the concentration of oxidized L-FABP in a sample to the total concentration of L-FABP in the sample (the sum total of oxidized L-FABP and unoxidized L-FABP). From the viewpoint of accuracy, the method for quantifying L-FABP according to the first aspect preferably further includes a step of calculating an oxidation rate, which almost corresponds to the rate of oxidized L-FABP to L-FABP in a sample, based on a measured value of the above L-FABP under the above condition that a difference in measurement sensitivity be small, and a measured value under the above condition that the measurement sensitivity of oxidized L-FABP be higher. The “oxidation rate of L-FABP” can almost correspond to the ratio of a measured value under the above condition that the measurement sensitivity of oxidized L-FABP be higher to a measured value of L-FABP (e.g. label intensity) under the above condition that a difference in measurement sensitivity between oxidized L-FABP and unoxidized L-FABP be small (e.g. an absorbance ratio (OD ratio) represented by the following formula):

OD value under the above condition that the measurement sensitivity of oxidized L-FABP be higher/OD value of L-FABP under the above condition that a difference in measurement sensitivity between oxidized L-FABP and unoxidized L-FABP be small.

In addition, the “oxidation rate of L-FABP” can be also represented, for example, by the following formula:

(aX+bY)(OD value)/total concentration of L-FABP (OD value) (in the above formula, a and b are a coefficient, X is the concentration of oxidized L-FABP, and Y is the concentration of unoxidized L-FABP). The coefficient a is preferably a coefficient representing the reactivity of an antibody to oxidized L-FABP, and the coefficient b is preferably a coefficient representing the reactivity of an antibody to unoxidized L-FABP.

The method for quantifying L-FABP according to the first aspect includes a step of quantifying the amount of oxidized L-FABP in a sample or a parameter value which correlates therewith, and the quantifying step is preferably a step of quantifying the oxidized L-FABP. This is because the “the amount of oxidized L-FABP” has higher accuracy than the quantified result of each of the “oxidation rate of L-FABP” and the “total concentration of L-FABP in a sample” does. The parameter which correlates with the amount of oxidized L-FABP is not the amount of oxidized L-FABP itself but a parameter calculated by converting a measured value (e.g. label intensity). Specifically, examples of the parameter include a measured value under the condition that the measurement sensitivity of oxidized L-FABP be higher than the measurement sensitivity of unoxidized L-FABP, the “oxidation rate of L-FABP”, and the like. The above concentration of oxidized L-FABP can be quantified from a product of the above oxidation rate, and a measured value of L-FABP (the total concentration of L-FABP in a sample) under the above condition that a difference in measurement sensitivity between oxidized L-FABP and unoxidized L-FABP be small.

In the method for quantifying L-FABP according to the first aspect, a calibration curve is made based on a relationship between the label intensity measured (e.g. absorbance, enzyme label intensity, fluorescence intensity, UV intensity, radiation intensity, etc.) and the amount of L-FABP (e.g. concentration), and the quantification may or may not be carried out based on the above calibration curve (e.g. by comparison).

### <<Quantification Kit>>

The second aspect of the present invention is a quantification kit, used for the method for quantifying L-FABP according to the first aspect, the kit including a substance which can quantify L-FABP. In the quantification kit according to the second aspect, examples of the substance which can quantify L-FABP include substances which quantify L-FABP based on e.g. enzyme immunoassay (EIA, ELISA), fluorescence enzyme immunoassay (FLEIA), chemiluminescent enzyme immunoassay (CLEIA), chemiluminescent immunoassay (CLIA), electrochemiluminescence immunoassay (ECLIA), fluorescent antibody method (FA), radioimmunoassay (RIA), western blotting (WB) or immunoblotting, and specifically an anti-L-FABP antibody is preferred.

The anti-L-FABP antibody used is not particularly restricted as long as it can recognize L-FABP, and may be a known antibody or an antibody which will be developed in the future. Examples thereof include an antibody which recognizes a site exposed to the outside by the above denaturing treatment, the above methionine oxidation or the like.

More particularly, the above quantitative means is preferably an assay using sandwich ELISA that combines two antibodies having different recognition sites to the antigen (L-FABP). The two antibodies having different recognition sites are as described above.

The above quantitative means preferably includes the above anti-L-FABP antibody as a reagent, more preferably further includes a labeled anti-L-FABP antibody, and may include an adsorption inhibitor (such as bovine serum albumin (BSA), casein, skim milk or polyethylene glycol), a pretreatment solution (such as any surfactant or any buffer), a reaction buffer (such as any buffer), a chromogenic substance (such as 3,3′5,5′-tetramethylbenzidine or hydrogen peroxide water) and the like as required. The amount of adsorption inhibitor included in the above quantitative means is not particularly restricted as long as the effects of the present invention are not lost, and is preferably 0.05 to 10 mass %.

The above quantitative means is preferably a kit using sandwich ELISA that combines two antibodies having different recognition sites to an antigen, and more preferably a kit using an anti-L-FABP antibody clone L on the solid phase and an anti-L-FABP antibody clone 2 as a labeled antibody.

The quantification kit according to the second aspect preferably has, when quantification is carried out by an anti-L-FABP antibody, a means for denaturing L-FABP by a surfactant before the quantification. The quantification kit according to the second aspect more preferably further has a means for denaturing the above L-FABP in a sample by a surfactant, and a means for quantifying L-FABP after the denaturing treatment. The above surfactant is as described above.

It is preferred that the quantification kit according to the second aspect further have a means for treating L-FABP or oxidized L-FABP in a sample by an immunoagglutination promoter (preferably a chaotropic reagent or an organic amine compound), and the above quantitative means be a means for quantifying L-FABP after the above treatment.

Examples of specific aspects when the quantification kit according to the second aspect is a kit using sandwich ELISA include a kit including the following (1) to (10):

(1) L-FABP antibody solid phase microplate • • • • • • wells coated with anti-human L-FABP mouse monoclonal antibody (e.g. derived from clone L-producing cell line),
(2) denaturing solution (e.g. any surfactant),
(3) immunoagglutination promoter solution (e.g. a chaotropic reagent, an organic amine compound),
(4) reaction buffer,
(5) enzyme labeled antibody • • • • • • peroxidase labeled anti-human L-FABP mouse monoclonal antibody (e.g. derived from clone 2-producing cell line),
(6) enzyme substrate solution,
(7) wash agent (such as any buffer or surfactant),
(8) reaction stop solution (such as 1 N sulfuric acid),
(9) standard buffer (such as any buffer), and
(10) L-type fatty acid binding protein standard.
The concentration of (10) L-type fatty acid binding protein standard is not particularly restricted and is, for example, 10 to 10000 ng/mL, preferably 50 to 5000 ng/mL, more preferably 100 to 1000 ng/mL, further preferably 200 to 800 ng/mL, and particularly preferably 300 to 600 ng/mL.

The quantification kit according to the second aspect preferably includes a protein storage buffer containing BSA in order to prevent protein adsorption. Examples thereof include a protein storage buffer described below.

### (Protein Storage Buffer)

10 mM phosphate buffer (pH 7.2), 150 mM NaCl, 1.0% BSA, 0.1% NaN₃

### <<Method for Testing Kidney Diseases>>

The third aspect of the present invention is a method for testing kidney diseases, including a step of promoting an antigen-antibody reaction, and quantifying L-FABP in urine collected from a subject (e.g. patient) under a condition that the measurement sensitivity of oxidized L-FABP be higher than that of unoxidized L-FABP. In addition, the fourth aspect of the present invention is a method for testing kidney diseases, including a step of quantifying the amount of oxidized L-FABP in urine collected from a subject or a parameter value which correlates therewith after promoting an antigen-antibody reaction, and the above quantifying step is preferably a step of quantifying the amount of the above oxidized L-FABP. The parameter which correlates with the amount of oxidized L-FABP is not the amount of oxidized L-FABP itself but a parameter calculated by converting a measured value (e.g. label intensity). Specifically, examples of the parameter include the above-described measured value under a condition that the measurement sensitivity of oxidized L-FABP be higher than the measurement sensitivity of unoxidized L-FABP, the above-described “oxidation rate of L-FABP”, and the like. The methods for testing kidney diseases according to the third and fourth aspects may or may not include a step of collecting urine from a subject. The methods for testing kidney diseases according to the third and fourth aspects may or may not include a step of detecting L-FABP in urine. In addition, the methods for testing kidney diseases according to the third and fourth aspects may or may not include at least one step selected from the group consisting of the following (A) and (B1) to (B4):

(A) a step of comparing the known normal range of the amount of oxidized L-FABP or a parameter value which correlates therewith, or the known range of the amount of oxidized L-FABP in a kidney disease or a parameter value which correlates therewith, and the amount of oxidized L-FABP in urine of a subject or a parameter value which correlates therewith, and determining which range the amount in the subject or parameter value which correlates therewith corresponds to,
(B1) a step of comparing the amount of oxidized L-FABP in a healthy subject or a parameter value which correlates therewith, and the amount of oxidized L-FABP in a subject or a parameter value which correlates therewith, and determining the contraction of chronic kidney disease when the above value of the latter is significantly higher than the above value of the former, in which the above amount of oxidized L-type fatty acid binding protein in a healthy subject or the value may be an amount or a value when the above subject had been previously healthy,
(B2) a step of comparing the amount of oxidized L-FABP in a healthy subject or a parameter value which correlates therewith, and the amount of oxidized L-FABP in a subject or a parameter value which correlates therewith, and determining the contraction of acute kidney injury when the above value of the latter is significantly lower than the above value of the former, in which the above amount of oxidized L-type fatty acid binding protein in a healthy subject or the value may be an amount or a value when the above subject had been previously healthy,
(B3) a step of comparing the amount of oxidized L-FABP in a patient with acute kidney injury or a parameter value which correlates therewith, and the amount of L-FABP in a subject, and determining the contraction of chronic kidney disease when the above value of the latter is significantly higher than the above value of the former, in which the above amount of oxidized L-type fatty acid binding protein in a patient with acute kidney injury or the value may be an amount or a value when the above subject had previously contracted acute kidney injury, and
(B4) a step of comparing the amount of oxidized L-FABP in a patient with chronic kidney disease or a parameter value which correlates therewith, and the amount of L-FABP in a subject, and determining the contraction of acute kidney injury when the above value of the latter is lower than the above value of the former, in which the above amount of oxidized L-type fatty acid binding protein in a patient with chronic kidney disease or the value may be an amount or a value when the above subject had previously contracted chronic kidney disease.

The methods for testing kidney diseases according to the third and fourth aspects may or may not include unoxidized L-FABP, may include a mixture of oxidized L-FABP and unoxidized L-FABP, and preferably includes a mixture of oxidized L-FABP and unoxidized L-FABP or oxidized L-FABP. In the methods for testing kidney diseases according to the third and fourth aspects, the above kidney disease is preferably at least one kidney disease selected from the group consisting of CKD and AKI, and AKI is more preferred. Specific examples and preferred examples of the method for measuring e.g. detecting or quantifying L-FABP or oxidized L-FABP include the same as described above for the <<method for quantifying L-FABP>>. In the methods for testing kidney diseases according to the third and fourth aspects, it is needless to say that the above testing for kidney diseases is used for the judgement of disease progression and reference for therapeutic strategy; however, the testing is preferably at least one testing selected from the group consisting of determining the degree of seriousness of kidney diseases, predicting the risk of developing kidney diseases, and monitoring kidney disease progression, and more preferably at least one testing selected from the group consisting of determining the degree of seriousness of kidney disease prognosis, predicting the prognosis of the risk of developing kidney diseases, and predicting prognosis by monitoring kidney disease progression.

In the method for testing kidney diseases according to the fourth aspect, the above quantification is preferably quantification under a condition that the measurement sensitivity of oxidized L-FABP be higher than the measurement sensitivity of unoxidized L-FABP. Specific examples and preferred examples of the condition that the measurement sensitivity of oxidized L-FABP be higher than the measurement sensitivity of unoxidized L-FABP include the same specific examples and preferred examples as described above for the

### <<Method for Quantifying L-FABP>>.

The methods for testing kidney diseases according to the third and fourth aspects preferably further include a step of quantifying the above L-FABP under a condition that a difference in measurement sensitivity between oxidized L-FABP and unoxidized L-FABP is smaller than the difference in measurement sensitivity under the condition that the measurement sensitivity of the oxidized L-FABP is higher than the measurement sensitivity of the unoxidized L-FABP. Specific examples and preferred examples of the condition that a difference in measurement sensitivity between oxidized L-FABP and unoxidized L-FABP is smaller include the same specific examples and preferred examples as described above for the

### <<Method for Quantifying L-FABP>>.

In the methods for testing kidney diseases according to the third and fourth aspects, a calibration curve is made based on a relationship between the label intensity measured (e.g. absorbance, enzyme label intensity, fluorescence intensity, UV intensity, radiation intensity, etc.) and the amount of L-FABP (e.g. concentration), and the quantification may or may not be carried out based on the above calibration curve (e.g. by comparison).

The fifth aspect of the present invention is a method for testing kidney diseases based on the amount of oxidized L-FABP in a subject or a parameter value which correlates therewith, the method including a step of comparing the known normal range of the amount of oxidized L-FABP or a parameter value which correlates therewith, or the known range of the amount of oxidized L-FABP in a kidney disease or a parameter value which correlates therewith, and the amount of oxidized L-FABP in urine of a subject or a parameter value which correlates therewith, and determining which range the above amount in the subject or parameter value which correlates therewith corresponds to.

The methods for testing kidney diseases according to the third to fifth aspects can be carried out at an area under the curve (AUC) in the analytical results of ROC (receiver operating characteristics) of preferably 0.650 or more, more preferably 0.700 or more, and further preferably 0.710 or more.

The methods for testing kidney diseases according to the third to fifth aspects can be based on the quantified results of only L-FABP, can evaluate CKD or AKI, and also can consistently evaluate CKD and AKI. The methods for testing kidney diseases according to the third to fifth aspects may or may not include a method for diagnosing kidney diseases. In addition, the present invention may relate to the methods for testing kidney diseases according to the third to fifth aspects, and a method for treating or preventing kidney diseases, including administering a therapeutic agent or preventive medicine for a kidney disease determined in the methods to a subject, or may not relate to the above. As the above therapeutic agent or preventive medicine for a kidney disease, at least one drug selected from the group consisting of therapeutic agents or preventive medicines for chronic kidney disease and therapeutic agents or preventive medicines for acute kidney injury is provided.

### <<Test Kit, Companion Diagnostic Agent and Kidney Disease Marker>>

The sixth aspect of the present invention is a test kit used for the method for testing kidney diseases according to the third or fourth aspect, the kit including a substance which can quantify L-FABP or oxidized L-FABP. The seventh aspect of the present invention is a companion diagnostic agent using the method for testing kidney diseases according to the third or fourth aspect, the agent including a substance which can quantify L-FABP or oxidized L-FABP. The eighth aspect of the present invention is a kidney disease marker used as a target to be quantified in the method for testing kidney diseases according to the third or fourth aspect, the marker including L-type fatty acid binding protein or oxidized L-type fatty acid binding protein. In the description and claims, the “companion diagnostic agent” means a diagnostic agent used in a testing performed before actually administering drugs in order to predict the effect of a pharmaceutical product on each patient with a kidney disease (e.g. CKD, AKI), the risk of adverse effects, and an adequate dosage. In the companion diagnostic agent according to the seventh aspect, the kidney disease is preferably at least one disease selected from the group consisting of CKD and AKI. Specific examples and preferred examples of the substance which can quantify L-FABP or oxidized L-FABP in the test kit according to the sixth aspect and the companion diagnostic agent according to the seventh aspect include the same as described above for the <<quantification kit>>.

The above quantitative means preferably includes the above anti-L-FABP antibody as a reagent, more preferably further includes a labeled anti-L-FABP antibody, and may include an adsorption inhibitor (such as bovine serum albumin (BSA), casein, skim milk or polyethylene glycol), a pretreatment solution (such as any surfactant or any buffer), a reaction buffer (such as any buffer), a chromogenic substance (such as 3,3′,5,5′-tetramethylbenzidine or hydrogen peroxide water) and the like as required. The amount of adsorption inhibitor included in the above quantitative means is not particularly restricted as long as the effects of the present invention are not lost, and is preferably 0.05 to 10 mass %.

When quantification is carried out by an anti-L-FABP antibody, the test kit according to the sixth aspect and the companion diagnostic agent according to the seventh aspect preferably have a means for denaturing L-FABP by a surfactant before the quantification. Specific examples and preferred examples of the means for denaturing L-FABP by a surfactant include the same as described above for the <<quantification kit>>.

It is preferred that the test kit according to the sixth aspect and the companion diagnostic agent according to the seventh aspect further have a means for treating L-FABP in urine by an immunoagglutination promoter (preferably a chaotropic reagent or an organic amine compound), and the above quantitative means be a means for quantifying L-FABP after the above treatment.

Examples of specific aspects when the test kit according to sixth aspect and the companion diagnostic agent according to the seventh aspect are a kit using sandwich ELISA include a kit including the (1) to (10) above for the <<quantification kit>>.

The test kit according to the sixth aspect and the companion diagnostic agent according to the seventh aspect preferably include a protein storage buffer containing BSA in order to prevent protein adsorption.

### EXAMPLES

The present invention will now be described in more detail by way of examples of the present invention described below. It should be noted, however, that the present invention is not limited thereto and can be performed with various applications without departing from the technical idea of the present invention.

### Reference Example 1

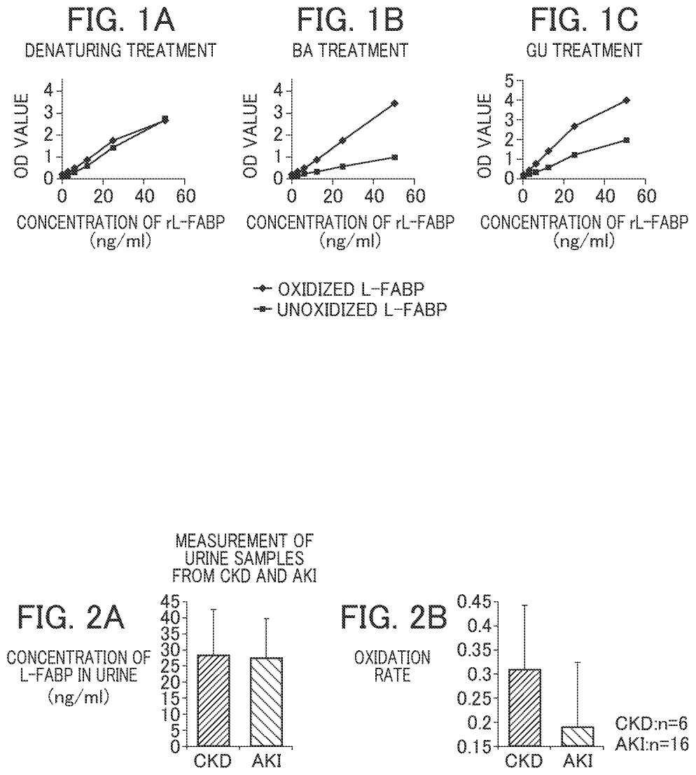

Oxidized recombinant L-FABP with various concentrations, treated with 50 mM AAPH at 37° C. for 60 minutes, and unoxidized recombinant L-FABP with various concentrations, which was not treated, were each denatured by 1 w/v % SDS at 25° C. for 10 minutes, and then subjected to ELISA using the antibody from “RENISCHEM L-FABP ELISA High Sensitivity Kit” (manufactured by CMIC HOLIDNGS CO., LTD.) to measure the color intensity (OD 450 nm) of the labeled antibody. The above test kit was used in accordance with the measurement method in the document usually appended. The results are shown in FIG. 1A.

On the other hand, ELISA was performed in the same manner as above except that the denaturing treatment was carried out by 1000 mM benzamidine hydrochloride in place of SDS at 25° C. for 10 minutes (hereinafter, referred to as “BA treatment”). The results are shown in FIG. 1B. In addition, ELISA was performed in the same manner as above except that the denaturing treatment was carried out by 1500 mM guanidinium chloride in place of SDS at 25° C. for 10 minutes (hereinafter, referred to as “GU treatment”). The results are shown in FIG. 1C.

As can be seen from the results shown in FIG. 1A, it is found that the OD value of oxidized recombinant L-FABP and the OD value of unoxidized recombinant L-FABP are almost equal at each concentration of recombinant L-FABP.

On the other hand, as can be seen from the results shown in FIGS. 1B and 1C, it is found that the OD measurement sensitivity of oxidized recombinant L-FABP is greater than the OD measurement sensitivity of unoxidized recombinant L-FABP at any one concentration of recombinant L-FABP when the BA treatment or the GU treatment is carried out in place of the denaturing treatment with SDS. It is thought that the reason why the measurement sensitivity becomes greater is because the inner region of L-FABP recognized by the antibody is exposed to the outside in oxidized L-FABP. On the other hand, it is thought that even when the anti-L-FABP antibody, which recognizes the inner region of L-FABP, is used for the measurement, structure changes, in which the inner region of L-FABP recognized by the above antibody is exposed to the outside, did not occur in unoxidized recombinant L-FABP, and thus measurement intensity does not increase.

### Example 1

Each urine sample from patients with chronic kidney disease (CKD) and patients with acute kidney injury (AKI) was denatured by 1 w/v % SDS at 25° C. for 10 minutes, and then the total concentration (ng/ml) of L-FABP in urine was measured using the antibody from “RENISCHEM L-FABP ELISA High Sensitivity Kit” (manufactured by CMIC HOLIDNGS CO., LTD.) The results are shown in FIG. 2A. In addition, each urine sample from patients with CKD (n=6) and patients with AKI (n=16) was subjected to ELISA in the same manner as above except that the BA treatment was carried out in place of the denaturing treatment with SDS. The oxidation rate of L-FABP in urine was calculated from the OD value after the BA treatment/the OD value after the denaturing treatment with SDS. The results are shown in FIG. 2B.

As can be seen from the results shown in FIG. 2A, it is found that the total concentration (ng/ml) of L-FABP in urine (including oxidized L-FABP and unoxidized L-FABP) is almost equal between patients with CKD and patients with AKI. On the other hand, as can be seen from the results shown in FIG. 2B, it is found that the oxidation rate of L-FABP in patients with AKI is significantly lower than that in patients with CKD. In addition, it can be said that these results show that when intracellular L-FABP is excreted to urine by acute kidney injury (AKI) regardless of a physiological condition, the proportion of unoxidized L-FABP is high, while L-FABP excreted to urine under a physiological condition has a high oxidation rate.

In addition, the concentration of oxidized L-FABP in urine of patients with CKD and patients with AKI can be quantified from a product of the oxidation rate of L-FABP in patients with CKD and patients with AKI shown in FIG. 2B, and the total concentration of L-FABP in urine of patients with CKD and patients with AKI shown in FIG. 2A, respectively.

### SEQUENCE LISTING

CMCF-006PCTseq.text

1. A method for quantifying L-type fatty acid binding protein, comprising: promoting an antigen-antibody reaction, and quantifying L-type fatty acid binding protein under a condition that measurement sensitivity of oxidized L-type fatty acid binding protein is higher than measurement sensitivity of unoxidized L-type fatty acid binding protein.

2. The method according to claim 1, wherein the condition is a condition formed by a treatment with a chaotropic reagent or an organic amine compound.

3. The method according to claim 1, further comprising: quantifying L-type fatty acid binding protein under a condition that a difference in measurement sensitivity between oxidized L-type fatty acid binding protein and unoxidized L-type fatty acid binding protein is smaller than the difference in measurement sensitivity under the condition that the measurement sensitivity of oxidized L-type fatty acid binding protein is higher than the measurement sensitivity of unoxidized L-type fatty acid binding protein.

4. The method according to claim 3, wherein the condition that a difference in measurement sensitivity is smaller is a condition formed by a denaturing treatment of the L-type fatty acid binding protein in a sample by a surfactant.

5. The method according to claim 3, further comprising: calculating an oxidation rate, which substantially corresponds to a rate of the oxidized L-type fatty acid binding protein to the L-type fatty acid binding protein in a sample, based on a measured value of the L-type fatty acid binding protein under the condition that the difference in measurement sensitivity is small and a measured value under the condition that the measurement sensitivity of the oxidized L-type fatty acid binding protein is high.

6. The method according to claim 1, comprising: using a kit comprising a substance which can quantify L-type fatty acid binding protein.

7. A method for testing a kidney disease, comprising: promoting an antigen-antibody reaction, and quantifying L-type fatty acid binding protein in urine of a subject under a condition that a measurement sensitivity of oxidized L-type fatty acid binding protein is higher than measurement sensitivity of unoxidized L-type fatty acid binding protein.

8. A method for testing a kidney disease, comprising: quantifying an amount of oxidized L-type fatty acid binding protein in urine of a subject or a parameter value which correlates therewith after promoting an antigen-antibody reaction.

9. The method according to claim 8, wherein the quantification is quantification under a condition that measurement sensitivity of oxidized L-type fatty acid binding protein is higher than measurement sensitivity of unoxidized L-type fatty acid binding protein.

10. The testing method according to claim 7, wherein the condition is a condition formed by a treatment with a chaotropic reagent or an organic amine compound.

11. The method according to claim 7, further comprising: quantifying the L-type fatty acid binding protein under a condition that a difference in measurement sensitivity between the oxidized L-type fatty acid binding protein and the unoxidized L-type fatty acid binding protein is smaller than the difference in measurement sensitivity under the condition that the measurement sensitivity of the oxidized L-type fatty acid binding protein is higher than the measurement sensitivity of the unoxidized L-type fatty acid binding protein.

12. The method according to claim 11, wherein the condition that the difference in measurement sensitivity is smaller is a condition formed by a denaturing treatment of the L-type fatty acid binding protein in urine by a surfactant.

13. The method according to claim 11, further comprising: calculating an oxidation rate, which substantially corresponds to a rate of the oxidized L-type fatty acid binding protein to the L-type fatty acid binding protein in urine, based on a measured value of the L-type fatty acid binding protein under the condition that a difference in measurement sensitivity is small and a measured value under the condition that a measurement sensitivity of the oxidized L-type fatty acid binding protein is high.

14. A method for testing a kidney disease based on an amount of oxidized L-type fatty acid binding protein in a subject or a parameter value which correlates therewith, the method comprising:
comparing a known normal range of an amount of the oxidized L-type fatty acid binding protein or a parameter value which correlates therewith, or a known range of an amount of the oxidized L-type fatty acid binding protein in a kidney disease or a parameter value which correlates therewith, and
an amount of the oxidized L-type fatty acid binding protein in urine of a subject or a parameter value which correlates therewith; and
determining in which range the amount in the subject or parameter value which correlates therewith is included.

15. The method according to claim 7, the comprising: using a test kit comprising a substance which can quantify the L-type fatty acid binding protein or the oxidized L-type fatty acid binding protein.

16. A companion diagnostic method, comprising: the method according to claim 7, and using a companion diagnostic agent comprising a substance which can quantify the L-type fatty acid binding protein or the oxidized L-type fatty acid binding protein.

17. The method according to claim 7, comprising: quantify a kidney disease marker comprising L-type fatty acid binding protein or oxidized L-type fatty acid binding protein.

18. The method according to claim 8, comprising: using a test kit comprising a substance which can quantify the L-type fatty acid binding protein or the oxidized L-type fatty acid binding protein.

19. A companion diagnostic method, comprising: the method according to claim 8, and using a companion diagnostic agent comprising a substance which can quantify the L-type fatty acid binding protein or the oxidized L-type fatty acid binding protein.

20. The method according to claim 8, comprising: quantify a kidney disease marker comprising L-type fatty acid binding protein or oxidized L-type fatty acid binding protein.

21. The method according to claim 14, comprising: using a test kit comprising a substance which can quantify the L-type fatty acid binding protein or the oxidized L-type fatty acid binding protein.

22. A companion diagnostic method, comprising: the method according to claim 14, and using a companion diagnostic agent comprising a substance which can quantify the L-type fatty acid binding protein or the oxidized L-type fatty acid binding protein.

23. The method according to claim 14, comprising: quantify a kidney disease marker comprising L-type fatty acid binding protein or oxidized L-type fatty acid binding protein.
